(19) United States
(12) Patent Application Publication
(10) Pub. No.: US 2025/0022700 A1
(43) Pub. Date: Jan. 16, 2025

(54) HIGH THROUGHPUT MASS SPECTRAL DATA GENERATION

(71) Applicant: DH Technologies Development Pte. Ltd., Singapore (SG)

(72) Inventors: Stephen A. TATE, Barrie (CA); Christopher LOCK, Richmond Hill (CA)

(73) Assignee: DH Technologies Development Pte. Ltd., Singapore (SG)

(21) Appl. No.: 18/714,355

(22) PCT Filed: Nov. 30, 2022
(86) PCT No.: PCT/IB2022/061619

§ 371 Date: May 29, 2024

Related U.S. Application Data
(60) Provisional application No. 63/285,766, filed on Dec. 3, 2021.

(51) Int. Cl. H01J 49/00; H01J 49/04

(52) U.S. Cl. CPC H01J 49/0036; H01J 49/0045; H01J 49/0431

(57) ABSTRACT

Methods and systems for mass spectrometry are disclosed. In one example, a method comprises: receiving, by a mass spectrometer via a sampling system operably connected thereto, at least one sample containing at least one known compound; modulat-ing at least one instrument parameter of the mass spectrometer through a plurality of instrument parameter values; analyzing the at least one sample while applying each of the plurality of instrument parameter values; acquiring a plurality of mass spectral (MS) datasets each corresponding to one of the applied plurality of instrument parameter values; encoding each of the plurality of MS datasets to generate a corresponding plurality of MS results each corresponding to one of the applied instrument parameter values; and compiling and storing the MS datasets and MS results in a spectral library in association with the applied instrument parameter values.

### CROSS-REFERENCE TO RELATED APPLICATION

This application is being filed on Nov. 30, 2022, as a PCT International Patent Application that claims priority to and the benefit of U.S. Provisional Application No. 63/285,766, filed on Dec. 3, 2021, which application is hereby incorporated by reference in its entirety.

### INTRODUCTION

Elucidation of chemical structure of compounds is a central task of sample analysis using mass spectrometry (MS). Conventionally, a sample containing an unknown compound is analyzed using a mass spectrometer to obtain a mass spectrum, and then the mass spectrum is compared to standard or reference mass spectra of a mass spectral library to determine the identity of the compound. However, due to the complexity of mass spectrometers, a small change of one single instrument parameter may cause significant change of the MS spectrum of the same compound analyzed by the mass spectrometer. Thus, it is highly desirable to optimize analytical methods and instrument parameters to obtain mass spectra with the best quality. However, optimization of instrument parameters is practically challenging when using traditional methods, in part because optimization often requires a large quantity of experiments under varied instrument parameters, which is time-consuming and inefficient.

In addition, many existing spectral libraries do not provide sufficient information regarding the instrument parameters used for the reference mass spectra, or only provide limited reference mass spectra obtained under certain or fixed instrument parameters. Thus, the existing spectral libraries are limited in data granularity and lack spectral dimensionality with respect to each reference compound. As an example, for compounds in complex mixtures, fragmentation patterns of their ions in tandem mass spectrometry are widely used for structural identification because these patterns can provide both the elemental composition of the compound as well as a direct read-out of labile bonds. However, the fragmentation pattern of a compound largely depends on instrument parameters that relate to the fragmentation (e.g., collision energy applied to the precursor ions) and/or the sample matrix (e.g., solvent, environment) that surrounds or interacts with the compound. Many existing spectral libraries do not provide a full spectrum of fragmentation patterns obtained under varied instrument parameters and/or varied sample matrices with respect to the reference compound. The lack of information regarding instrument parameter variation of the reference mass spectra may significantly undermine the accuracy of compound identification when using library search and comparison.

### SUMMARY

In one aspect, the present disclosure relates to a method for mass spectrometry. In one example, a method comprises: receiving, by a mass spectrometer via a sampling system operably connected thereto, at least one sample containing at least one known compound; modulating at least one instrument parameter of the mass spectrometer through a plurality of instrument parameter values; analyzing the at least one sample while applying each of the plurality of instrument parameter values; acquiring a plurality of mass spectral (MS) datasets each corresponding to one of the applied plurality of instrument parameter values; encoding each of the plurality of MS datasets to generate a corresponding plurality of MS results each corresponding to one of the applied instrument parameter values; and compiling and storing the MS datasets and MS results in a spectral library in association with the applied instrument parameter values. In some embodiments, the MS results comprise a at least one mass spectrum generated for each instrument parameter value. In some embodiments, the at least one sample comprises a plurality of different samples.

In some embodiments, the modulating the at least one instrument parameter comprises at least one of: modulating through a plurality of instrument parameter values while analyzing a single sample; or modulating through a plurality of instrument parameter values while analyzing across a plurality of samples; or modulating through a plurality of instrument parameter values while analyzing each sample of a plurality of samples.

In some embodiments, the modulating the at least one instrument parameter comprises setting a plurality of instrument parameter values ramped in a range for a single instrument parameter; and the at least one sample is analyzed under at least one of the plurality of values. In some embodiments, the plurality of values ramped in the range provides a granularity of the at least one instrument parameter and has an increment from about 0.01% to about 10% of the range.

In some embodiments, encoding the plurality of MS datasets comprises converting the MS results into a suitable format for use by a machine learning algorithm. In some embodiments, encoding the plurality of MS datasets further comprises: transmitting at least a portion of the MS results to a computing device, the computing device comprising the machine learning algorithm and a processor for processing the MS results; and training the machine learning algorithm with the MS results. In some embodiments, encoding the plurality of MS datasets further comprises: determining, for the at least one known compound, a relationship of the MS results with the at least one instrument parameter using the machine learning algorithm.

In some embodiments, the encoding step further comprises: extracting at least one spectral feature from the plurality of MS datasets; and vectorizing the extracted spectral feature to generate at least one spectral vector, and the method further comprises: identifying a relationship between the at least one spectral vector and the instrument parameter; or determining an impact of the modulation on the spectral vector. In some embodiments, the method further comprise: performing a principle component analysis (PCA) on the at least one spectral vector. In some embodiments, extracting at least one spectral feature comprises at least one of: calculating the total ion intensity of the mass spectra; annotating/identifying/grouping MS peaks of the mass spectra; calculating m/z values, peak area, and intensities of MS peaks; determining a relationship between related MS peaks; extracting a spectral feature indicative of a fragmentation pattern; identifying precursor ions and product ions; or extracting a spectral feature indicative of a sample matrix.

In some embodiments, the method further comprises: analyzing a test sample containing at least one target analyte to obtain a test result of the test sample; comparing the test result with the spectral library; and predicting an identity of the at least one analyte in the test sample. In some embodiments, the method further comprises: training a machine learning algorithm with the MS results; and applying the machine learning algorithm in the comparing or predicting operations.

In some embodiments, the sampling system is a high-throughput sampling system; and each of the plurality of samples is introduced to the mass spectrometer and analyzed by the mass spectrometer in a total time less than about 20 seconds, or less than about 5 seconds.

In some embodiments, the sampling system comprises an Acoustic Droplet Ejector (ADE) operably coupled to an Open Port Interface (OPI); and each of the plurality of samples is ejected from a sample volume by the ADE and introduced to the mass spectrometer through the OPI.

In some embodiments, the at least one instrument parameter comprises at least one of: a collision energy (CE); an electron energy; a parameter related to fragmentation; a parameter related to ionization; a parameter related to the introduction of ions to a quadrupole ion guide; or a parameter that controls an ion mobility device.

In some embodiments, the mass spectrometer comprises an ionization source, a collision cell, and an ion detector; and the collision cell comprises at least one fragmentation module selected from: collision induced dissociation (CID), surface induced dissociation (SID), electron capture dissociation (ECD), electron transfer dissociation (ETD), metastable-atom bombardment, photo-fragmentation, or combinations thereof.

In some embodiments, the at least one instrument parameter comprises a fragmentation parameter that controls the fragmentation module; and the plurality of instrument parameter values comprises a plurality of fragmentation parameter values. In some embodiments, the method further comprises: producing precursor ions of each sample in the ionization source; transmitting the precursor ions of each sample into the collision cell; generating fragment ions from the precursor ions of each sample in the collision cell under each of the applied modulated fragmentation parameter; and detecting the precursor and fragment ions using the ion detector, wherein the MS results comprise at least one MSMS spectrum generated for each fragmentation parameter value.

In some embodiments, the method further comprises: generating, from the MSMS spectra, a plurality of fragmentation results each corresponding to one of the applied fragmentation parameter values, wherein the plurality of fragmentation results comprise at least one of: a spectral feature indicative of the precursor and fragment ions for each sample; a fragmentation pattern of each sample analyzed under each one of the applied fragmentation parameter values; or a fragmentation pathway for the at least one known compound.

In some embodiments, the method further comprises: training a machine learning algorithm with the plurality of fragmentation results; and determining, for the at least one known compound, a relationship of the plurality of fragmentation results with the fragmentation parameter, using the machine learning algorithm.

In some embodiments, the method further comprises: generating a plurality of MS results for a plurality of known compounds by repeating the method described herein; and compiling and storing the plurality of MS results corresponding to the plurality of known compounds in the spectral library.

In another example, a method for mass spectrometry comprises: receiving, by a mass spectrometer via a sampling system operably connected thereto, at least one sample containing at least one known compound, wherein the mass spectrometer comprises an ionization source, a collision cell comprising at least one fragmentation module, and an ion detector; modulating at least one fragmentation parameter of the mass spectrometer through a plurality of fragmentation parameter values; analyzing the at least one sample while applying each of the plurality of fragmentation parameter values; producing precursor ions of each sample in the ionization source; transmitting the precursor ions of each sample into the collision cell; generating fragment ions from the precursor ions of each sample in the collision cell under each of the applied fragmentation parameter values; detecting the precursor and fragment ions of each sample using the ion detector; acquiring a plurality of mass spectral (MS) datasets each corresponding to one of the applied plurality fragmentation parameter values; encoding each of the plurality of MS datasets to generate a corresponding plurality of MS results each corresponding to the applied fragmentation parameter value, wherein the MS results comprise at least one MSMS spectrum generated for each fragmentation parameter value; generating, from the MSMS spectra, a plurality of fragmentation results each corresponding to one of the applied fragmentation parameter values; and compiling and storing the MS datasets and MS results in a spectral library in association with the applied fragmentation parameter values.

In another aspect, the present disclosure relates to a system for mass spectrometry. In one example, a system comprises: a mass spectrometer; a high-throughput sampling system operative to introduce a plurality of samples to the mass spectrometer; a processor operatively coupled to the high-throughput sampling system and the mass spectrometer; and memory, coupled to the processor, the memory storing instructions that, when executed by the processor, perform operations comprising: receiving, by a mass spectrometer via a sampling system operably connected thereto, at least one sample containing at least one known compound; modulating at least one instrument parameter of the mass spectrometer through a plurality of instrument parameter values; analyzing the at least one sample while applying each of the plurality of instrument parameter values; acquiring a plurality of mass spectral (MS) datasets each corresponding to one of the applied plurality of instrument parameter values; encoding each of the plurality of MS datasets to generate a corresponding plurality of MS results each corresponding to one of the applied instrument parameter values; and compiling and storing the MS datasets and MS results in a spectral library in association with the applied instrument parameter values.

In yet another aspect, the present disclosure relates to a computer program product. In one example, a computer program product comprises a non-transitory computer-readable storage medium whose contents include a program with instructions being executed on a processor so as to perform a method, the method comprising: processing a plurality of MS results; and determining, for at least one known compound, a relationship of the MS results with the modulated instrument parameter. The plurality of MS results is generated by: receiving, by a mass spectrometer via a sampling system operably connected thereto, at least one sample containing at least one known compound; modulating at least one instrument parameter of the mass spectrometer through a plurality of instrument parameter values; analyzing the at least one sample while applying each of the plurality of instrument parameter values; acquiring a plurality of mass spectral (MS) datasets each corresponding to one of the applied plurality of instrument parameter values; and encoding each of the plurality of MS datasets to generate a corresponding plurality of MS results each corresponding to one of the applied instrument parameter values.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

FIG. 1 is a schematic diagram illustrating one exemplary system in accordance with various aspects and embodiments of the present disclosure.

FIG. 2 depicts a block diagram of a computing device.

FIG. 3 is a schematic diagram illustrating one particular example system in accordance with various aspects and embodiments of the present disclosure.

FIG. 4 is a flow diagram illustrating one particular example method for mass spectrometry in accordance with various aspects and embodiments of the present disclosure.

FIG. 5A is a flow diagram illustrating one particular example of one operation of FIG. 4, in accordance with various aspects and embodiments of the present disclosure.

FIG. 5B is a flow diagram illustrating one particular example of one operation of FIG. 4, in accordance with various aspects and embodiments of the present disclosure.

FIG. 5C is a flow diagram illustrating one example method involving training and applying a machine learning algorithm for compound identification.

FIG. 6 is an exemplary schematic diagram illustrating one particular example workflow employing the present teachings, in accordance with various aspects and embodiments of the present disclosure.

FIG. 7 is an exemplary schematic diagram illustrating another particular example workflow employing the present teachings, in accordance with various aspects and embodiments of the present disclosure.

FIG. 8 is an exemplary schematic diagram illustrating yet another particular example workflow employing the present teachings, in accordance with various aspects and embodiments of the present disclosure.

FIG. 9 is a flow diagram illustrating another particular example method for mass spectrometry in accordance with various aspects and embodiments of the present disclosure.

FIG. 10A is a flow diagram illustrating one particular example of one operation of FIG. 9, in accordance with various aspects and embodiments of the present disclosure.

FIG. 10B is a flow diagram illustrating one particular example of one operation of FIG. 9, in accordance with various aspects and embodiments of the present disclosure.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the terminology used herein is for the purpose of description and should not be regarded as limiting.

### DETAILED DESCRIPTION

### Definitions and Interpretations for Selected Terms

For the purposes of interpreting this specification, the following definitions will apply and whenever appropriate, terms used in the singular will also include the plural and vice versa. The definitions set forth below shall supersede any conflicting definitions in any documents incorporated herein by reference.

As used herein, the singular forms “a,” “an,” and “the,” include both singular and plural referents unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements or method steps. It will be appreciated that the terms “comprising,” “comprises,” and “comprised of” as used herein comprise the terms “consisting of,” “consists,” and “consists of.”

The recitation of numerical ranges by endpoints includes all numbers and fractions subsumed within the respective ranges, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6, or ≥7, etc. of said members, and up to all said members.

Unless otherwise defined, all terms used in the present disclosure, including technical and scientific terms, have the meaning as commonly understood by one of ordinary skill in the art to which this disclosure belongs. By means of further guidance, term definitions are included to better appreciate the teaching of the present disclosure.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure, and form different embodiments, as would be understood by those in the art.

Further, in describing various embodiments, the specification may have presented a method or process or workflow as a particular sequence of steps. However, to the extent that the method or process or workflow does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process and/or workflow should not be limited to the performance of their steps in the order written, and one skilled in the art can readily appreciate that the sequences may be varied and still remain within the spirit and scope of the various embodiments.

In the following detailed description, references are made to the accompanying drawings that form a part hereof, and in which are shown by way of illustrations, specific embodiments, or examples. Embodiments may be practiced as methods, systems, devices, or workflows. Accordingly, embodiments may take the form of a hardware implementation, an entirely software implementation, or an implementation combining software and hardware aspects. The following detailed description is, therefore, not to be taken in a limiting sense, and the scope of the present disclosure is defined by the appended claims and their equivalents.

### System for Mass Spectrometry

The present disclosure relates generally to systems, methods, processes, workflows, and computer software for mass spectrometry, including but not limited to: sample preparation and delivery, sample analysis, high-throughput mass spectrometry, modulation of instrument parameter, mass spectral data generation, mass spectral analysis and data processing, spectral feature extraction, spectral entry and spectral library construction, prediction of compound identity, analyte identification, construction of machine learning model/algorithm training and improving the machine learning algorithm, and application of the machine learning model or algorithm.

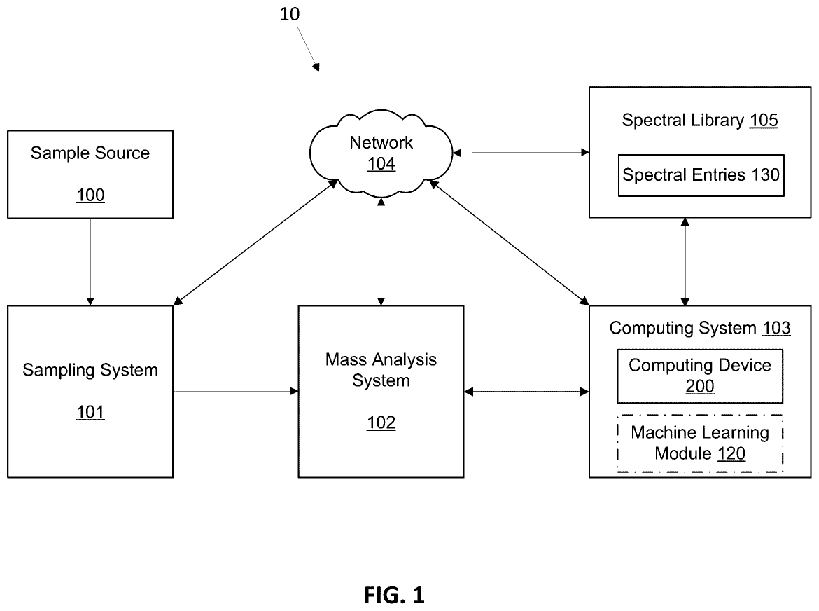

Now referring to FIGS. 1-4, examples of the present system for mass spectrometry and various components and aspects thereof will be illustrated and described. FIG. 1 illustrates a schematic diagram of one example system in accordance with various embodiments. In the illustrated example, the system 10 includes at least one of the following subsystems or components: a sample source 100, a sample preparation and delivery system (“sampling system”) 101, a mass analysis system 102, a computing system 103, a network 104, and a spectral library 105. The subsystems of the system 10 may be operably interconnected between or among each other to allow transmission of the electric signals through the entire system 10 of a part thereof.

The sample source 100 includes one or more samples. In some examples, the sample source is a collection or pool of samples each housed in a well of a well plate. In some examples, the sample source contains pluralities of collections of samples, the samples containing at least one compound with a known structure or identity (reference sample), or at least one unknown compound (test sample). A reference sample to be analyzed by the system 10 may include at least one target compound of high purity/quality. The at least one known compound may be used as a reference or standard compound and include one single compound of known chemical structure or a plurality of target compounds derived from a compound family such as isomers. In some examples, the at least one compound includes one or more target analytes to be identified.

The sample to be analyzed by the system 10 may be prepared by any suitable technique. In some examples, the sample may also include a sample matrix that contains everything but the target compounds. For example, the sample matrix may contain a solvent, an impurity, a contaminant, one or more compounds from the environment (e.g., blood, urine, cell culture medium, etc.) where the sample is derived from, an interfering compound, a degradation product of the target compound, a deterioration product of the target compound, an internal reference or standard, one or more assisting agents that are added to the sample to assist in sample analysis. In some examples, the sample is free or substantially free from biological or environmental matrices. In some examples, the sample source 100 includes a plurality of samples each having the same known compound and different sample matrix that varied across the plurality of samples. In some examples, the sample source 100 includes a plurality of samples each having a different known compound and the same sample matrix. In some examples, the sample source 100 includes a plurality of samples each having a different known compound and a different sample matrix.

The sampling system 101 of FIG. 1 is operative to receive the sample from the sample source and transport and deliver the sample in appropriate form to the mass analysis system 102. In one embodiment, the sampling system 101 is a high-throughput sampling system. In a non-limiting example, the introduction of a sample to the mass analysis system 102 takes less than 20 seconds in a high-throughput sampling system. In another non-limiting example, the introduction of the sample takes less than 10 seconds. And in yet another non-limiting example, the introduction of the sample takes less than 5 seconds. In some embodiments, each sample of the sample source 100 is introduced to the mass analysis system 102 and analyzed by the mass analysis system 102 in a total period of time that is less than about 20 seconds, less than about 10 seconds, less than about 5 seconds, or less than about 2 seconds. Generally, the high-throughput sampling system enables the analysis of a large quantity of samples and generates a large volume of mass spectral (MS) data within a relatively short period of time (e.g., one day). Non-limiting examples of the high-throughput system include, but are not limited to: acoustic droplet ejection (ADE) open-port interface (OPI) mass spectrometry (MS) system (hereinafter ADE-OPI-MS), RapidFire, etc.

The mass analysis system 102 may include a mass spectrometer operative to perform at least one of the functions: receive samples from the sampling system 101, ionize the samples to produce ion species of the samples, fragment the ion species to produce production ions, filter and detect selected ions of interest from the sample ions, and analyze the detected ions to produce MS signals and data for the analyzed samples.

The computing system 103 of FIG. 1 includes computing resources, components, and modules that are collectively operable to perform various functions including but not limited to: communicating with other components of the system 10, receiving and transmitting electrical signals with other components, receiving, responding to, and executing user instructions, performing calculations, processing raw mass spectrometry data received from the mass analysis system 102, analyzing and processing mass spectrometry data, generating and analyzing mass spectra for the samples, identifying, annotating, and assigning MS peaks of mass spectra, extracting spectral features from mass spectra, conducting spectral searching and spectral comparison, identifying analytes, predicting compound or structural identity, and outputting analytical report to end users.

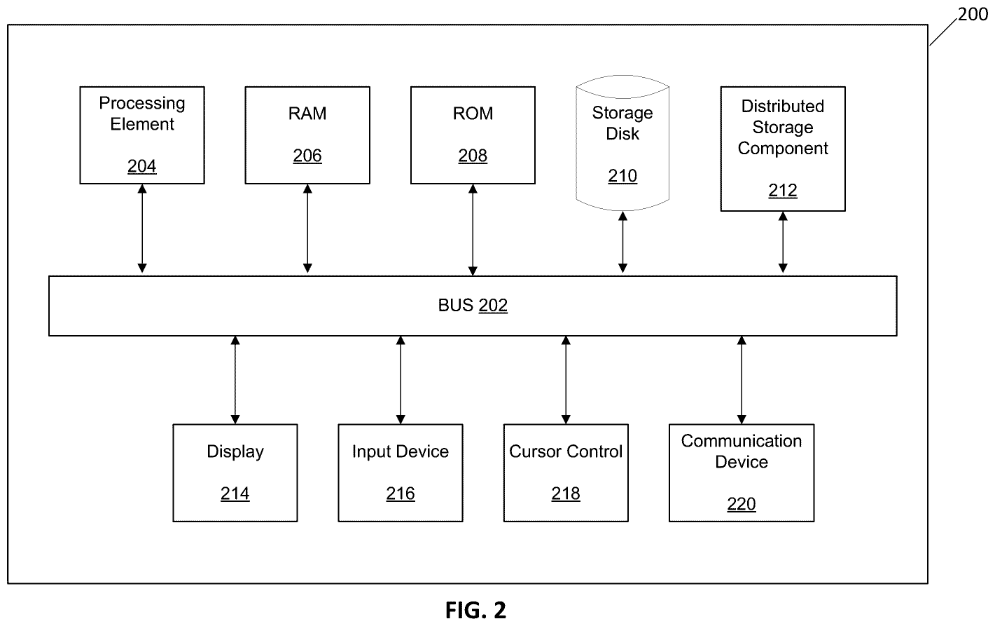

The computing system 103 may include a machine learning module 120 operative to execute a machine learning model or algorithm, and/or to train the machine learning model or algorithm with MS results. In one embodiment, the machine learning module 120 can include one or more processing units, storage units, network interfaces, or other hardware and software elements. The term ““module” is intended to refer to computer-related entities, comprising either hardware, a combination of hardware and software, software, or software in execution. For example, a module can be implemented as a process running on a processor, such as processing element 204 in FIG. 2, a hard disk drive, multiple storage drives (of optical and/or magnetic storage medium), a program, and/or a computer. By way of illustration, both an application running on a server and the server can be a module. One or more modules can reside within a process and/or a thread of execution, and a module can be localized on one computer and/or distributed between two or more computers as desired for a given implementation. For example, the machine learning module 120 can include a neural network executing on a computing device that perform the operations described herein.

The machine learning model is created using a machine learning algorithm. The machine learning model may be created by a computing device (e.g., computing device 200 of FIG. 2) or the processing element 204 thereof using standard techniques such as training and test MS results. In some embodiments, the machine learning module further includes a machine learning model training unit, a machine learning model test unit, an analyte identification prediction unit, and an evaluation unit. The machine learning model is the set of parameters specific to a particular machine learning algorithm that can achieve an optimal classification of results. In various embodiments, the machine learning algorithm uses a support vector machine (SVM) algorithm or a decision tree algorithm to create the machine learning model.

The computing system 103 may include at least one computing device (e.g., computing device 200 shown in FIG. 2). It is noted that the computing system 103 may include a single computing device or may include a plurality of distributed computing devices in operative communication with components of a mass analysis system 102 or other subsystems of the system 10.

FIG. 2 illustrates one example of a computing device. In the illustrated example, the computing device 200 may include a bus 202 or other communication mechanism of similar function for communicating information, and at least one processing element 204 (collectively referred to as processing element 204) coupled with bus 202 for processing information. As will be appreciated by those skilled in the art, the processing element 204 may comprise a plurality of processing elements or cores, which may be packaged as a single processor or in a distributed arrangement. Furthermore, a plurality of virtual processing elements 204 may be included in the computing device 200 to provide the control or management operations for the mass analysis system 102.

The computing device 200 may also include one or more volatile memory (ies) 206, which can for example include random access memory (ies) (RAM) or other dynamic memory component(s), coupled to one or more busses 202 for use by the at least one processing element 204. Computing device 200 may further include static, non-volatile memory (ies) 208, such as read only memory (ROM) or other static memory components, coupled to busses 202 for storing information and instructions for use by the at least one processing element 204. A storage component 210, such as a storage disk or storage memory, may be provided for storing information and instructions for use by the at least one processing element 204. As will be appreciated, the computing device 200 may comprise a distributed storage component 212, such as a networked disk or other storage resource available to the computing device 200.

The computing device 200 may be coupled to one or more displays 214 for displaying information to a user. Optional user input devices 216, such as a keyboard and/or touchscreen, may be coupled to a bus for communicating information and command selections to the at least one processing element 204. An optional graphical input device 218, such as a mouse, a trackball or cursor direction keys for communicating graphical user interface information and command selections to the at least one processing element. The computing device 200 may further include an input/output (I/O) component, such as a serial connection, digital connection, network connection, or other input/output component for allowing intercommunication with other computing components and the various components of the mass analysis system 102.

In various embodiments, computing device 200 can be connected to one or more other computer systems via a network to form a networked system. Such networks can for example include one or more private networks or public networks, such as the Internet. In the networked system, one or more computer systems can store and serve the data to other computer systems. The one or more computer systems that store and serve the data can be referred to as servers or the cloud in a cloud computing scenario. The one or more computer systems can include one or more web servers, for example. The other computer systems that send and receive data to and from the servers or the cloud can be referred to as client or cloud devices, for example. Various operations of the mass analysis system 102 may be supported by operation of the distributed computing systems.

The computing device 200 may be operative to control operation of the components of the mass analysis system 102 and the sampling system 101 through a communication device 220, and to handle data generated by components of the mass analysis system 102 through the data processing system 300. In some examples, analysis results are provided by computing device 200 in response to the at least one processing element 204 executing instructions contained in memory 206 or 208 and performing operations on data received from the mass analysis system 102. Execution of instructions contained in memory 206 and/or 208 by the at least one processing element 204 can render the mass analysis system 102 and associated sample delivery components operative to perform methods described herein.

The term “computer-readable medium” as used herein refers to any media that participates in providing instructions to the processing element 204 for execution. Such a medium may take many forms, including but not limited to, non-volatile media, volatile media, and transmission media. Non-volatile media includes, for example, optical or magnetic disks, such as disk storage 210. Volatile media includes dynamic memory, such as memory 206. Transmission media includes coaxial cables, copper wire, and fiber optics, including the wires that comprise bus 202.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to the processing element 204 for execution. For example, the instructions may initially be carried on the magnetic disk of a remote computer. The remote computer can load the instructions into its dynamic memory and send the instructions over a telephone line using a modem. A modem local to computing device 200 can receive the data on the telephone line and use an infra-red transmitter to convert the data to an infra-red signal. An infra-red detector coupled to bus 202 can receive the data carried in the infra-red signal and place the data on bus 202. Bus 202 carries the data to memory 206, from which the processing element 204 retrieves and executes the instructions. The instructions received by memory 206 and/or memory 208 may optionally be stored on storage device 210 either before or after execution by the processing element 204.

In accordance with various embodiments, instructions operative to be executed by a processing element to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

Referring back to FIG. 1, the system 10 may include a network 104 operably connected to any one or all of the subsystems or components in the system 10. The network 104 is a communication network. The network 104 may be any suitable type of network and/or a combination of networks. The network 104 may be wired or wireless and of any communication protocol. The network 104 may include, without limitation, the Internet, a local area network (LAN), a wide area network (WAN), a wireless LAN (WLAN), a mesh network, a virtual private network (VPN), a cellular network, and/or any other network that allows the computing system 103 to operate as described herein. In the illustrated embodiment, the network 104 is a wireless local area network (WLAN).

The system 10 may further include at least one spectral library 105 operative to receive, compile, and store MS results, compound information, and spectral data generated from sample analysis. The spectral library 105 may include a plurality of spectral entries 130 with respect to each reference or standard compound. Each spectral entry 130 may include compound spectral data, compound details, sample metadata, and analytical metadata. The compound details may include chemical knowledge of the reference compound such as name, chemical formula, elemental composition, neutral mass, etc. The compound spectral data may include mass spectra of the reference compound, spectral features extracted from the mass spectra, secondary results derived from post-analysis data processing, etc. Sample metadata may include information regarding sample matrix (e.g., Mammalian such as tissues, blood, plasma etc., bacteria, virus, plants, water), sample preparation information, and additional information (e.g., sample location, storage conditions). The analytical metadata may include instrument type, instrument setting, instrument parameter, operating condition, modulation of instrument parameter and/or operating condition, sample injection mode, separation technique, consumable details (e.g., solvents, chemicals, sample tubes), etc. In some embodiments, the computing system 103 is operative to perform a library search using the spectral library 105 and/or to compare mass spectra of a test sample to the retrieved data from the spectral library 105 (such as molecular mass information or spectral features) to facilitate mass analysis and/or identification of compound identification. In some embodiments, the machine learning algorithm(s) included in the machine learning module 120 may be used to perform the library search, comparison, and prediction of compound identity.

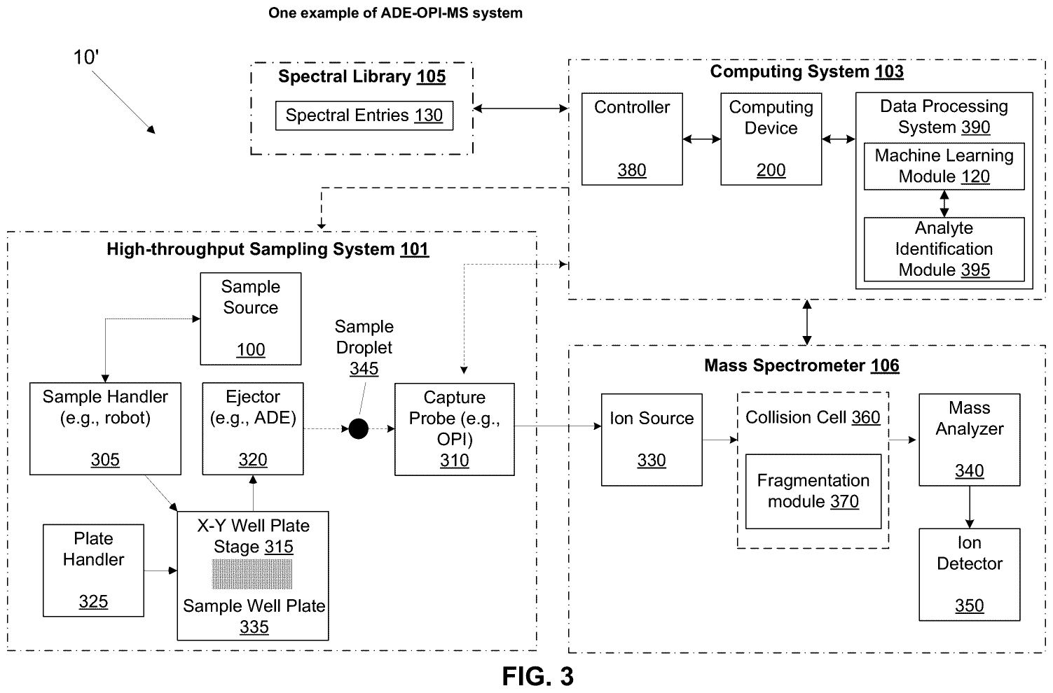

FIG. 3 illustrates a schematic view of another particular example system in accordance with various embodiments described herein. In the illustrated example, the system 10′ is operative to perform high-throughput mass spectrometry analysis. Similar to the system 10 of FIG. 1, the system 10′ includes a sampling system 101, a mass spectrometer 106, a computing system 103, and optionally a spectral library 105.

The sampling system 101 may include at least one of: a sample source 100, a sample handler 305, a capture probe 310, a X-Y well plate stage 315, an ejector 320, and a plate handler 325. The sample source 100 and the sample handler 305 are operative to retrieve collections of samples from the sample source 100 and to deliver the retrieved collections to capture locations associated with sample capture probes 310. The system 10′ may be operative to independently capture selected ones of the pluralities of samples at the capture locations from the pluralities of samples, to optionally dilute the samples and to transfer the captured samples to mass spectrometer 106 for mass analysis. In some embodiments, the sample source 100 may include a set of well plates in a storage housing and/or liquid for adding to well plates. The sample source 100 may include part of a liquid handling system that manipulates and/or injects liquid into the well plates. The sample handler 305 includes one or more electro-mechanical devices (e.g., robotics, conveyor belts, stages, etc.) that are capable of transferring the samples (e.g., well plates) from the sample source to other components of the sampling system 101 and/or to other components, such as the ejector 320 and/or the capture probe 310. As an example, the sample handler 305 may transfer a well plate 335 to the ejector 320 or the plate handler 325.

The ejector 320 is operable to eject droplets 345 from the wells of the well plate 335. The size of the droplet is typically from 1 to 10 nanoliter. The ejector 320 may be any type of suitable ejector, such as an acoustic ejector, a pneumatic ejector, or other type of contactless ejector. In an example, the plate handler 325 receives a well plate 335 from the sample handler 305. The plate handler 325 transports the well plate 335 to a capture location that may be aligned with the capture probe 310. Once in the capture location, the ejector 320 ejects droplets 345 from one or more wells of the well plate 335. The plate handler 325 may include one or more electro-mechanical devices, such as a translation stage that translates the well plate 335 in an X-Y plane to align wells of the well plate 335 with the ejector 320 and/or or the capture probe 310.

The mass spectrometer 106 includes at least one of: an ion source (e.g., ionization source) 330, a mass analyzer 340, an ion detector 350, and a collision cell 360. The mass spectrometer 106 can be operative, for example, through use of ion source(s) or generator(s) 330 produce sample ions of the sample introduced into the mass spectrometer 106. The collision cell 360 is operative to fragment the precursor ions produced by the ion source 330 to generate product ions (fragment ions) derived from the precursor ions. The mass spectrometer 106 is further operative to filter and detect selected ions of interest from the sample ions through the use of the mass analyzer 340 and ion detector 350. The mass analyzer 340 is operative to analyze the sample ions and produce a mass spectrometry (MS) dataset comprising all ion current signals from the sample ions.

In some embodiments, the mass spectrometer 106 is operative to perform tandem mass spectrometry analysis through the use of the collision cell 360. The collision cell 360 may further include a fragmentation module 370 operative to apply an energy to the selected precursor ions and cause the selected precursor ions to undergo fragmentation and generate product ions. The fragmentation module may include at least one of: collision induced dissociation (CID), surface induced dissociation (SID), electron capture dissociation (ECD), electron transfer dissociation (ETD), metastable-atom bombardment, photo-fragmentation, or combinations thereof.

It will also be appreciated by a person skilled in the art and in light of the teachings herein that the mass analyzer 340 can have a variety of configurations. Generally, the mass analyzer 340 is operative to process (e.g., filter, sort, dissociate, detect, etc.) sample ions generated by the ion source 330. By way of non-limiting example, the mass analyzer 340 can be a triple quadrupole mass spectrometer, or any other mass analyzer known in the art and modified in accordance with the teachings herein. Other non-limiting, exemplary mass spectrometer systems that can be modified in accordance with various aspects of the systems, devices, and methods disclosed herein can be found, for example, in an article entitled “Product ion scanning using a Q-q-Q linear ion trap (Q TRAP) mass spectrometer,” authored by James W. Hager and J. C. Yves Le Blanc and published in Rapid Communications in Mass Spectrometry (2003; 17:1056-1064); and U.S. Pat. No. 7,923,681, entitled “Collision Cell for Mass Spectrometer,” the disclosures of which are hereby incorporated by reference herein in their entireties.

Other configurations, including but not limited to those described herein and others known to those skilled in the art, can also be utilized in conjunction with the systems, devices, and methods disclosed herein. For instance, other suitable mass spectrometers include single quadrupole, triple quadrupole, Time of Flight (TOF), trap, and hybrid analyzers. It will further be appreciated that any number of additional elements can be included in the system 10 or 10′ including, for example, an ion mobility spectrometer (e.g., a differential mobility spectrometer) that is disposed between the ion source 330 and the ion detector 350 and is operative to separate ions based on their mobility difference between in high-field and low-field). Additionally, it will be appreciated that the mass analyzer 340 can comprise the ion detector 350 that can detect the ions that pass through the mass analyzer 340 and can, for example, supply a signal indicative of the number of ions per second that are detected.

The computing system 103 may include a computing device 200 as described above, a controller 380, and a data processing system 390. The controller 380 may be in the form of electronic signal processors and in electrical communication with other subsystems within the system 10′. The controller 380 may be operative to coordinate some or all of the operations of the pluralities of the various components of the system 10′. In one example, the controller 380 may be a controller for the mass spectrometer 106 and may be used as the primary controller for controlling components in addition to those components housed within the mass spectrometer 106. As such, the controller 380 may be considered the main or central controller that orchestrates, or communicates with, the other controllers to carry out the operations discussed herein in a more efficient manner.

The data processing system 390 may include various components and modules operative to process mass spectrometry data and to provide real-time feedback to users and other subsystems. In some embodiments, the data processing system 390 comprises a machine learning module 120 as described earlier. In some embodiments, the data processing system 390 further includes an analyte identification module 395 operable connected to the machine learning module 120. The analyte identification module 395 is operative to perform a library search and predict compound identity of a target analyte in a test sample, optionally through use of the trained machine learning algorithm.

In operation, the sampling system 101 (including sample source 100 and the sample handler 305) can iteratively deliver independent samples from a plurality of samples (e.g., a sample from a well of a well plate 335) to the capture probe 310. The capture probe 310 can dilute and transport each such delivered sample to the mass spectrometer 106 disposed downstream of the capture probe 310 for ionizing the diluted sample. The mass analyzer 340 can receive generated ions from the ion source 330 and/or the collision cell 360 for mass analysis. The mass analyzer 340 is operative to selectively separate ions of interest from generated ions received from the ion source 330 and to deliver the ions of interest to the ion detector 350 that generates a mass spectrometer signal indicative of detected ions to the computing system 103. In some aspects, the separate ions of interest may be indicated in an analysis instruction associated with that sample. In some aspects, the separate ions of interest may be indicated in an analysis instruction identified by an indicia physically associated with the plurality of samples.

The system 10′ may include a commercial product such as a Biomek computer available from Beckman Coulter Life Sciences, which is in operative communication with a mass spectrometer 106 and a controller for the capture probe 310, which may include, for example, an a SciexOS® or Analyst® computer available from AB Sciex. The Analyst® or SciexOS® computer includes a control controller for the capture probe 310, represented for example by Sciex open port probe (OPP) (also referred to as OPI) software, and a controller for the mass spectrometer 106, which may be the Analyst® computer. The mass spectrometer 106 and the controller for capture probe 310 may be further in operative communication with an ejector 320 and an X-Y Well Plate Stage 315, which may be, for example, a liquid droplet ejector with embedded computer or processor. For the purposes of this disclosure, these distributed controller components may collectively be considered to be a system controller, and depending upon the configuration may be centralized, or distributed as is the case here. For instance, one of the controllers or controller components may send signals to the other controllers to control the respective devices.

In one particular example, the high-throughput system 10′ employs the ADE-OPI-MS technology. The ADE-OPI-MS system according to the present disclosure relies on acoustic dispensing of droplets directly from the wells of the plate under analysis. The acoustically dispensed droplets, which are at nanoliter scale, with the precise control and independent of the sample solvent, are acoustically ejected from the ejected sample and introduced to a vortex at the opening of the OPI and delivered directly to the ionization source of the MS for detection. The extremely small samples required, coupled with the method's resilience in handling unpurified samples, make this technology ideally suited for direct sampling from the well plate. The ADE-OPI-MS system and method also offer significant speed advantages: with an average analysis time of 1-2 seconds per sample and a small quantity of 1-10 nanoliter per sample, such that a typical well plate containing 384 wells can be analyzed in under 15 min. Thus, the ADE-OPI-MS system advantageously enables high-throughput analysis of a large quantity of samples and generate a large volume of data within a meaning time frame such as a day. In addition, the ADE-OPI is compatible with both nominal and high resolution mass spectrometers, allowing rapid quantification with the former, and extensive analyte identification with the latter. More examples of the ADE-OPI-MS system and various components thereof can be found in U.S. Pat. No. 10,770,277, the disclosure of which is incorporated by reference herein in its entirety.

### Method for Mass Spectrometry

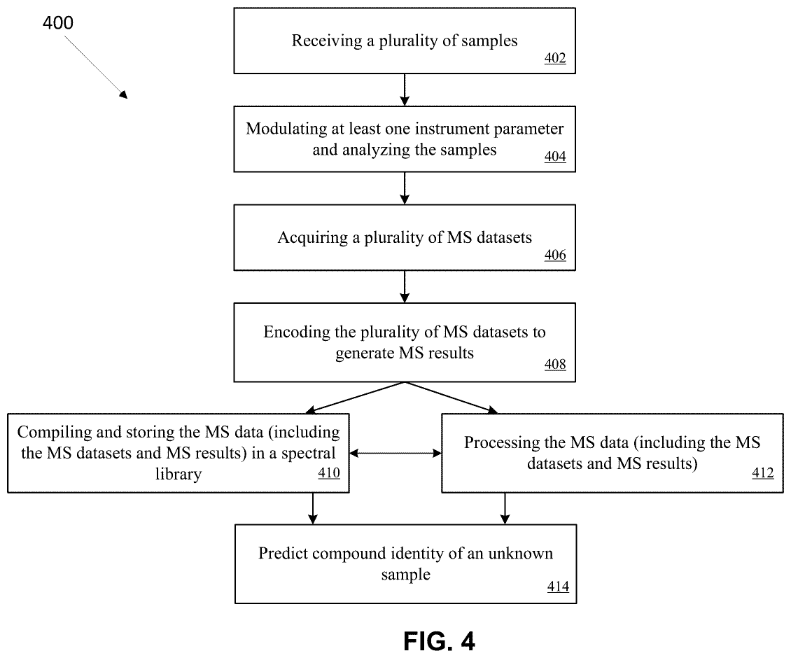

FIG. 4 illustrates a flow diagram of one example method 400 for mass spectrometry, in particular, generating MS data and building a MS spectral library for at least one known compound. In the illustrated example, the method 400 includes at least one operation of 402, 404, 406, 408, 410, 412, and 414. For the sole purpose of convenience, method 400 will be described through use the example system 10 or 10′. However, it is appreciated that the method 400 may be performed by any suitable system.

Operation 402 includes receiving by the mass spectrometer 106, via the sampling system 101, at least one sample containing at least one known compound. In some embodiments, the at least one sample comprises a single sample housed in a well of the well plate 335. In some embodiments, the at least one sample comprises a plurality of samples respectively housed in a plurality of wells of at least one well plate 335. At operation 402, the system 10′ may be controlled by the controller 380 to introduce the at least one sample from the well plate 335 to the mass spectrometer 106 through use of the ejector 320 (such as ADE) and the capture probe 310 (such as OPI). In some embodiments, a plurality of samples may be successively introduced to the mass spectrometer 106 through use of the ADE and OPI.

Operation 404 includes modulating at least one instrument parameter of the mass spectrometer 106 through a plurality of instrument parameter values and analyzing the plurality of samples with the mass spectrometer 106 while applying each of the plurality of instrument parameter values to the mass spectrometer 106. At operation 404, the computing system 103 is operative to select at least one instrument parameter, generate a modulation, and apply the modulation to the selected instrument parameter. The instrument parameter used herein encompasses the parameters that control any constituent component, device, or subsystem of the system 10 or 10′. The instrument parameter also encompasses operating conditions such as temperature, pressure, environment, gas resource, under which the system 10′ is operated. Non-limiting examples of the instrument parameter include: scan time, resolution, collision gas, curtain gas, exit potential, ion source gas, ion spray voltage, ion spray temperature, collision energy (CE); electron energy; a parameter related to fragmentation; a parameter related to ionization; a parameter related to the introduction of ions to a quadrupole ion guide; or a parameter that controls an ion mobility device.

At operation 404, at least one selected instrument parameter is modulated. In some embodiments, the modulating the at least one instrument parameter comprises modulating through a plurality of instrument parameter values while analyzing a single sample. In some embodiments, the modulating the at least one instrument parameter comprises modulating through a plurality of instrument parameter values while analyzing across a plurality of samples. In some embodiments, the modulating the at least one instrument parameter comprises modulating through a plurality of instrument parameter values while analyzing each sample of a plurality of samples.

In some embodiments, operation 404 includes varying one instrument parameter by setting a plurality of values ramped in a range for the one instrument parameter, and at least one sample is analyzed under at least one of the plurality of values, through use of the mass spectrometer 106. The at least one sample may be a single sample or a plurality of different samples. Ramping the plurality of values provides a granularity of the at least one instrument parameter and has an increment or gradient from about 0.01% to about 10% of the range. As one example, the at least one instrument parameter comprises an ionspray voltage (IV). Modulation of the ion spray voltage includes setting 100 values (IV₁, IV₂, . . . , IV₁₀₀) ramped in a range from 1,000 V to 6,000 V, with an increment of 50 V for the ramped values (IV₁=1,000 V, IV₂=1,050 V, IV₃=1,100 V, . . . , IV₁₀₀=6,000 V). The modulation results in a relatively high granularity or parameter density reflected by a small increment of 1% of the entire range (50 V/5,000 V). If a single sample is analyzed under each of the 100 IV values when they are applied to the mass spectrometer, the modulation of IV is across a single sample. If each one of a plurality of samples (e.g., 100 samples) is analyzed at one single IV value of the 100 IV values, the modulation of IV is across the plurality of samples.

In some embodiments, if ramping is not an available option for an instrument parameter, then modulation includes setting a plurality of “single-shot” modes for the instrument parameter and analyzing the at least one sample at each “single shot” of the instrument parameter.

In some examples, operation 404 includes modulating a plurality of instrument parameters. Each of the plurality of instrument parameters may be modulated through a plurality of the instrument parameter values and analyzed while applying each of the plurality of instrument parameter. The plurality of instrument parameter values for each instrument parameter may be varied across a single sample or multiple samples of a plurality of samples. As an example, two instrument parameters (P₁, P₂) may be respectively modulated at operation 404 to generate M values ramped in a first range for P₁ and N values ramped in a second range for P₂. The modulation may result in a total number M×N combinations of P₁ and P₂ and produce M×N MS results corresponding to each combination of P₁ and P₂. In some embodiments, the sample may be analyzed under each value of the modulated P₁ with a fixed value of P₂; and under each value of the modulated P₂ with a fixed value of P₂.

Operation 406 includes acquiring a plurality of MS datasets each corresponding to one of the applied plurality of instrument parameter values. Each MS dataset includes raw data generated from the sample analyzed under each instrument parameter value.

Operation 408 includes encoding and/or processing the plurality of MS datasets. At operation 408, the MS dataset generated by the mass spectrometer 106 is transmitted to the computing system 103 and encoded and/or processed by the data processing system 390 to generate a corresponding plurality of MS results each corresponding to one of the applied instrument parameter values. The plurality of MS results includes at least one mass spectrum of the sample analyzed at each specific instrument parameter value of the modulated instrument parameter.

Operation 408 may further include extracting at least one spectral feature from the plurality of MS datasets and MS results. The extracting at least one spectral feature includes but is not limited to: calculating the total ion intensity of the mass spectra; annotating and/or identifying and/or grouping MS peaks of the mass spectra; calculating m/z values, peak area, and intensities of MS peaks; determining a relationship between related MS peaks; calculating a mass difference between two related MS peaks, extracting a spectral feature indicative of a fragmentation pattern; calculating a total intensity of fragment ions, identifying precursor ions and product ions; or extracting a spectral feature indicative of a sample matrix, or combinations thereof.

Operation 408 may further include vectorizing the extracted spectral features to generate at least one spectral vector that represent at least one spectral feature. An example spectral vector is the total intensity of the fragment ions produced by the collision cell 360 of the system 10′. A plurality of vectors may be generated at 408 to represent various spectral features extracted from the mass spectra with respect to each analyzed sample. The vector may be in a form of a variable having a plurality of values, with each value corresponding to a specific instrument parameter value applied to the mass spectrometer. For example, vectorizing the total ion intensity of the fragment ions produces a vector comprising 100 values of the total ion intensity corresponding to 100 varied CE values, at each of which the sample is fragmented and analyzed.

Operation 410 includes transmitting MS data (collectively referring to all data related to or generated from execution of the method 400, including but not limited to the MS datasets and MS results) to a spectral library operative to compile and store the MS data and MS results therein. The spectral library may include a plurality of spectral entries, each spectral entry including the MS datasets, MS results, mass spectra, extracted spectral features, vectors, and compound information in a form of reference MS data for each reference compound. Depending on the granularity of the modulated instrument parameter, the spectral library may advantageously have a high data density and provide a broad and comprehensive profile of reference MS data generated at varied instrument parameter values of the modulated instrument parameters.

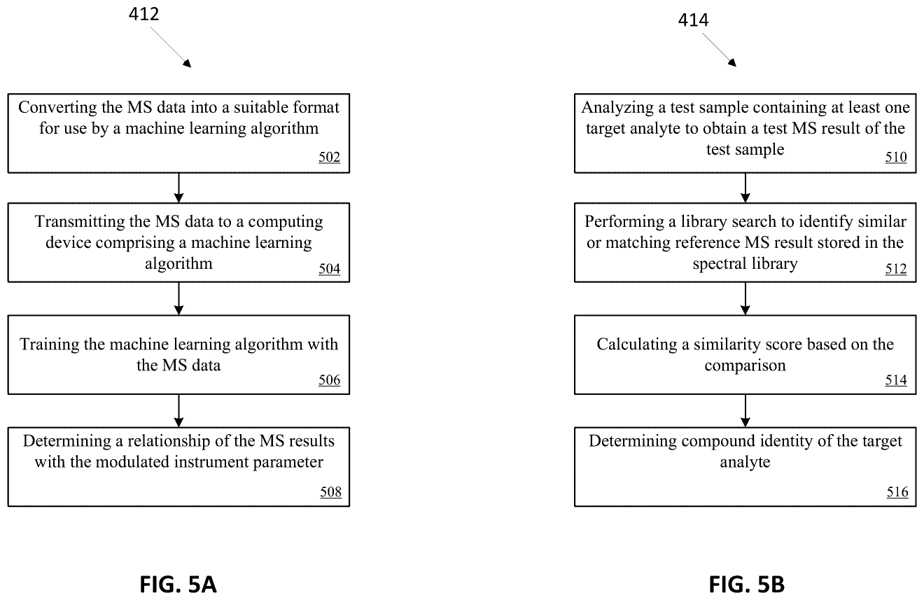

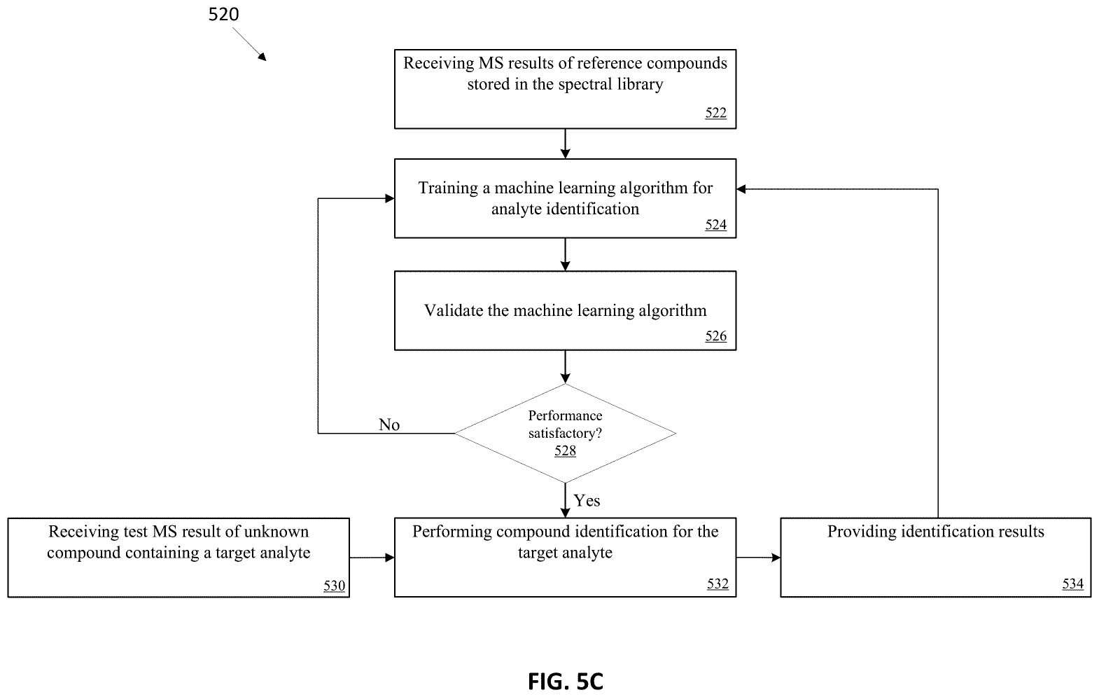

Operation 412 includes processing the MS data. At operation 412, the MS datasets and MS results corresponding to the instrument parameter values may be further processed through use of the data processing system 390. FIG. 5A illustrates an exemplary flow diagram of operation 412. In the illustrated example, operation 412 includes at least one of operations 502, 504, 506, and 508. At operation 502, the MS results are converted into a suitable format for use by the machine learning module 120. In a non-liming example, the MS results are converted into vectors that are processed by one or more machine learning algorithms (or models).

Operation 504 includes transmitting the MS data in the suitable format to a computing system or device comprising a machine learning algorithm, e.g., the computing system 103 comprising the machine learning module 120. Operation 506 includes training the machine learning algorithm with the MS results and/or building a machine learning algorithm. Operation 508 includes determining a relationship of the MS results with the instrument parameter.

At operation 508, one or more machine learning algorithms may be used to correlate the MS results with the varied values of the modulated instrument parameters. For example, a vector representing the total ion intensity is correlated to the varied values of ionspray voltage (IV) for the known compound of the analyzed sample using the machine learning algorithm(s). The machine learning algorithm is further trained with a plurality of vectors representing different spectral features generated from the MS results. At operation 508, the machine learning algorithm(s) may be used to identify a relationship between the at least one spectral vector and the instrument parameter; and/or determine an impact of the modulation on the spectral vector. The machine learning algorithm(s) may be trained with MS results from a large quantity of known compounds with similar or analogous structures.

The trained machine learning algorithm may be used to predict a relationship between the MS result (e.g., total ion intensity) and the instrument parameter (e.g., IV) for a unknown compound, suggest an optimal value of the instrument parameter to be used to generate a mass spectrum of desired quality for analyzing the unknown compound, and/or predict the identity of structure of the unknown compound.

Operation 508 may further include performing a principle component analysis (PCA) on the at least one spectral vector to determine the relationship between the spectral vector and the instrument parameter and/or the correlation between or among multiple instrument parameters. As an example, PCA may be performed to determine a relationship between the total ion intensity and each of a plurality of modulated instrument parameters such as collision energy (CE) and modulated ionspray voltage (IV), and identify the primary factor or co-factors determinative of the fragmentation pathway of the precursor ions. More examples of the implementation of PCA can be found in U.S. Pat. No. 9,442,887, the disclosure of which is incorporated herein by reference. The PCA may be performed through use of the machine learning algorithm trained by the machine learning module 120.

Referring back to FIG. 4, the method 400 may further include operation 414. Operation 414 may be performed in addition to, or as an alternative to, operation 508. Operations 414 and 508 may involve use of the analyte identification module 395 of the system 10′. Operation 414 includes at least one of: analyzing a test sample containing at least one target analyte to obtain a test MS result of the test sample; comparing the test MS result with the spectral library; and predicting an identity of the at least one analyte in the test sample. Operation 414 may further include training a machine learning algorithm with the MS results; and applying the machine learning algorithm in the comparing or predicting operations. For example, the trained machine learning algorithm from operation 412 may be used to perform operation 414.

FIG. 5B illustrates a flow diagram of a particular example of operation 414. In the illustrated example, operation 414 includes at least one of operations 510, 512, 514, and 516. At operation 510, a test sample containing at least one unidentified target analyte or an unknown compound of interest is analyzed by a mass spectrometer to obtain a test MS result of the test sample. The test sample may be analyzed under a modulated instrument parameter to produce a plurality of test mass spectra obtained at the varied value of the modulated instrument parameter. At operation 512, a library search is performed to compare the test mass spectra of the test sample to the reference mass spectra of the reference compounds stored in the spectral library with respect to each value of the modulated instrument parameter. At operation 514, an overall similarity or matching score may be calculated based on the comparison at each value of the modulated instrument parameter. At operation 516, a matching reference compound from the spectral library is identified based on the similarity score, and the identity of the unknown compound may be determined. Comparing with the traditional library search that disregards the impact of instrument parameter, the present spectral library takes the granularity of instrument parameters into account and provides a high data density for the reference mass spectral data. The present method thus enables a comprehensive spectral comparison across a range of the instrument parameter values, increases dimensionality of spectral comparison in compound identification, and improves the confidence and accuracy of compound identification.

FIG. 5C illustrates a flow diagram of an example method for training and applying a machine learning algorithm for predicting compound identity of a target analyte in an unknown sample. The method 520 is additional or alternative to operations 412 and 414, and may be performed in conjunction with the method 400 or any operation thereof. In the illustrated example, the method 520 includes at least one of operations 522, 524, 526, 528, 530, 532, and 534. Operation 522 includes retrieving search results from a library search (e.g., from operation 512) and receiving reference MS data stored in the spectral library. In some examples, the library search results store a plurality of reference MS spectra and extracted/vectorized spectral features corresponding to one or more modulated instrument parameters for each reference compound.

Operation 524 includes training a machine learning algorithm for compound identification based on the spectral library search results. In some examples, the machine learning algorithm is trained using a supervised machine learning training method. In other examples, the machine learning algorithm is trained using an unsupervised machine learning training method. Examples of possible machine learning methods include support vector machines, weighed voting systems, neural networks, k-nearest neighbors, decisions tress, and logistic regression. The machine learning training method is used to process this data and generate a machine learning algorithm. In some embodiments, the training data is preprocessed into a training set and a test/validation set. In some of these embodiments, these steps are repeated with different training sets. Training a machine learning algorithm includes integrating multiple variables associated with the modulated instrument parameter and extracted/vectorized spectral features. This data can include one or more analytes identified as being present in a sample. The data can also be stored with labels identifying whether the identified analyte is a true positive, true negative, false positive, or a false negative. In some examples, these labels are provided by an expert user. In other examples, an algorithm is used to assign these labels.

In some embodiments, the training of the machine learning algorithm is an iterative process. For example, iterations of training a machine learning algorithm are carried out to produce an optimized machine learning algorithm. In some examples, if the evaluation of the machine learning algorithm fails to meet set benchmarks or thresholds the process the machine learning algorithm will go through additional training. When a machine learning algorithm fails the evaluation step the iterative process may include adding new variables or removing variables which are used to train a new machine learning algorithm. In some examples, a machine learning algorithm is further refined as more MS data are collected. For example, the machine learning algorithm can go through the training process with new features to create a machine learning algorithm which is updated based on additional samples. In other examples, a plurality of machine learning algorithms are trained with a multi-model approach.

In some embodiments, the iterative process starts with using new MS data to perform an analyte library search. In other examples, the iterative process starts with selecting different variables from the spectral library search results or performing an additional search with adjusted settings. In further examples, the iterative process includes adjusting the training for the machine learning module 120. Combinations of the above adjustments can be made and additional adjustments to the training data or machine learning algorithms are also possible.

Operation 526 includes validating the trained machine learning algorithm. In some embodiments, a trained machine learning algorithm is validated by analyzing a plurality of known samples with the machine learning algorithm to generate predictions and comparing the predictions with compound identities of the plurality of known samples. Operation 528 includes determining whether the trained machine learning algorithm perform satisfactorily. In some embodiments, if the machine learning algorithm is not successfully validated at the operation 528, the method 520 iterates by repeating the operations 522, 524, and 526. Other methods of adjusting the machine learning algorithm may also be used. Once a trained machine learning algorithm is validated it may be used for compound identification of unknown samples, e.g., through use of the analyte identification module 395. Operation 530 includes receiving test MS results from analysis of a test sample containing an unidentified target analyte. Operation 532 includes performing compound identification. Operation 534 includes providing the identification results back to the machine learning algorithm to continue to training the machine learning algorithm and/or to provide reinforcement training.

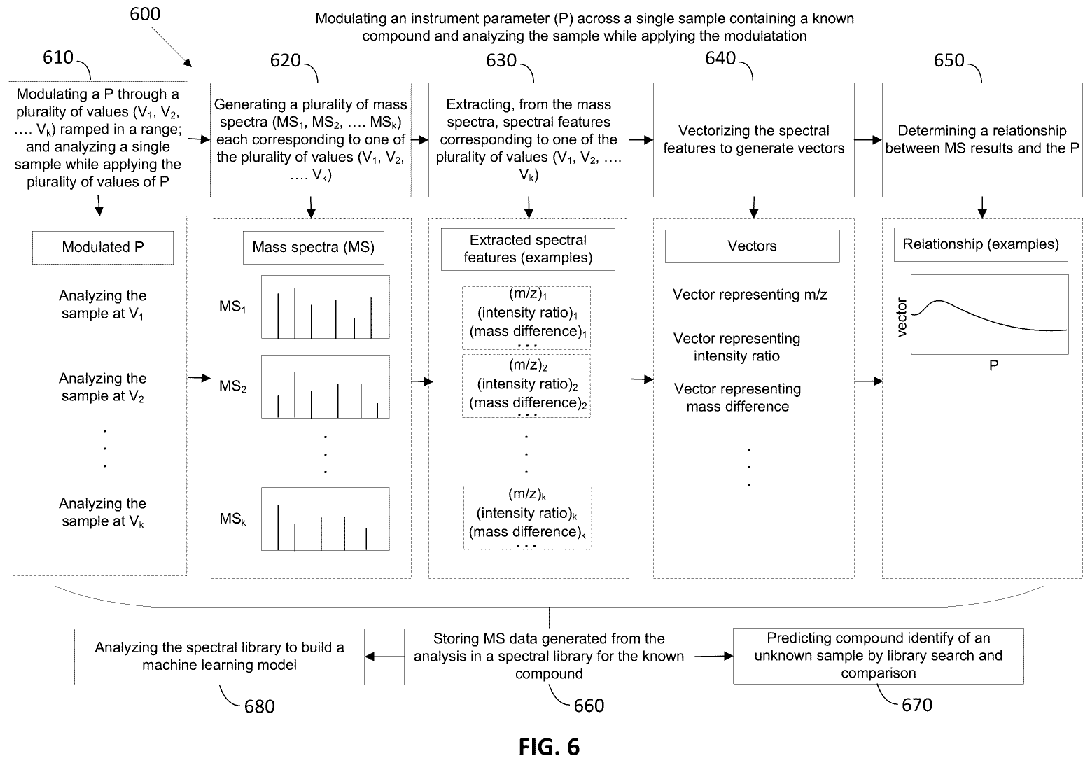

Now referring to FIGS. 6-8, particular example workflows derived from the method 400 will be illustrated and described. FIG. 6 illustrates a schematic view of an exemplary workflow related to modulation of an instrument parameter (P) across a single sample containing a known compound. In the illustrated example, the method 600 includes operations 610, 620, 630, 640, 650, 660, and optionally 670 and 680.

At operation 610, an instrument parameter (P) is modulated and a number of k values (V₁, V₂, V₃, . . . , Vₖ) varied and ramped in a range is set for the P across a single sample containing a known compound. A single sample is analyzed while each of the k values (V₁, V₂, V₃, . . . , Vₖ) of the P is applied to the mass spectrometer. At operation 620, a plurality of MS results (MS₁, MS₂, MS₃, . . . , MSₖ) is generated. Each MS result may include one or more mass spectra corresponding to one of the k values of the modulated P. At operation 630, at least one spectral feature (collectively referred to as spectral feature) are extracted from each of the mass spectra. Non-limiting examples of the spectral feature includes m/z value of characteristic MS peaks, mass difference of related peaks, total intensity of fragment ions, intensity ratios of fragment ions to precursor ions, etc. In some embodiments, a plurality of spectral features are extracted from the mass spectra. At operation 640, each extracted spectral feature is vectorized to generate a spectral vector representing that spectral feature. At operation 650, the MS results are further processed to determine, for the known compound, a relationship between the spectral feature and the varied values of the modulated P, an impact of the modulation on each vector, or an impact factor (significance) of the P on each vector. Various analytical tools, mathematical models, or fitting algorithms may be used at 650 to facilitate data processing. At operation 660, the MS data (including the MS datasets and MS results) related to and/or generated from the analysis and data processing are stored in a spectral library as a form of reference MS data for the known compound. The spectral library accordingly includes a plurality of reference mass spectra generated under varied values of the modulated instrument parameter for the known compound.

At operation 670, the spectral library is used by the analyte identification module 395 to predict compound identity of a test sample containing an unknown compound. At operation 680, the spectral library is further analyzed to build or improve a machine learning algorithm and train or improve the machine learning algorithm with the reference data stored in the spectral library through use of the machine learning module 120. The trained machine learning algorithm may be used to determine the relationship between MS results and the instrument parameter at operation 650, to optimize instrument parameter or operating condition for analysis of an unknown compound, and/or to predict identify of the unknown compound at operation 670.

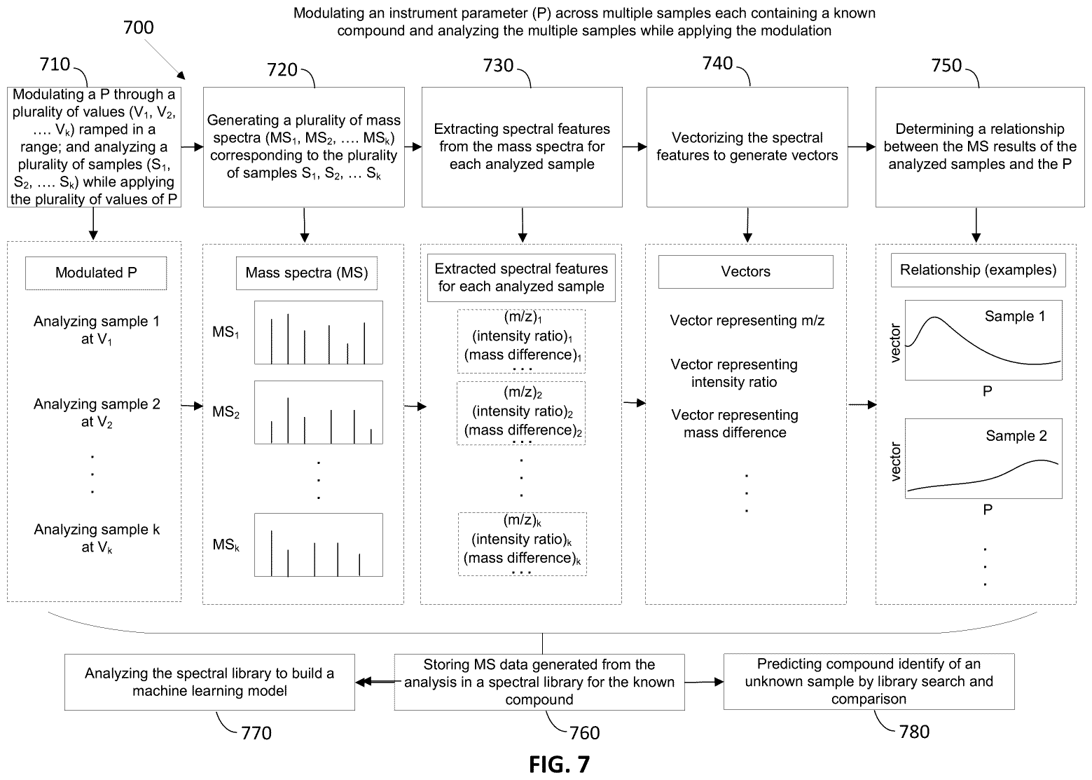

FIG. 7 illustrates a schematic view of another exemplary workflow. The workflow 700 is a variation of the workflow 600 and related to modulation of an instrument parameter (P) across multiple samples each containing at least a known compound. The multiple samples may each contain the same known compound but a different sample matrix (e.g., solvent, impurity, biological environment). Alternatively, the multiple samples may each contain a different compound from others. In some embodiments, the compounds may be of the similar kind or share a common structural feature of interest. In some embodiments, the multiple samples each contain a different compound with a known identity. A large quantity of samples may be analyzed using the workflow 700.

In the illustrated example, the method includes operations 710, 720, 730, 740, 750, 760, and optionally 770 and 780. At operation 710, an instrument parameter (P) is modulated and a number of k values (V₁, V₂, V₃, . . . , Vₖ) varied and ramped in a range is set for the P across a plurality of samples (S₁, S₂, S₃, . . . , Sₖ) each containing at least one known compound. Each of the k samples is respectively analyzed under each of the corresponding k values (V₁, V₂, V₃, . . . , Vₖ). At operation 720, a plurality of MS results (MS₁, MS₂, MS₃, . . . , MSₖ) is generated. Each MS result may include one or more mass spectra each corresponding to one of the k values of the modulated P. In some embodiments, the P is modulated across each one of a plurality of samples, and each of the plurality of samples (S₁, S₂, S₃, . . . , Sₖ) is analyzed whiling applying the plurality of values (V₁, V₂, V₃, . . . , Vₖ) of the P. A plurality of MS results (MS₁, MS₂, MS₃, . . . , MSₖ) is obtained for each one of the plurality of samples (S₁, S₂, S₃, . . . , Sₖ).

At operation 730, spectral features are extracted from each of the mass spectra. At operation 740, the extracted spectral features are vectorized to generate a plurality of spectral vectors each representing the a specific spectral feature. At operation 750, the MS results are further processed to determine, for the known compound of each sample, a relationship between the spectral features and the varied values of the modulated P, an impact of the modulation on each vector, or an impact factor (significance) of the P on each vector. If the multiple samples contain different sample matrices, an impact of sample matrix on the vector and a correlation between the sample matrix and the modulated instrument parameter may also be determined at operation 750. If the multiple samples contain compounds of similar type or structural features, a common relationship for the similar compounds may be generalized at operation 750. At operation 760, the generated MS data (including the MS datasets and MS results) are stored in a spectral library as a form of reference MS data for the known compound. The spectral library accordingly includes a plurality of reference mass spectra generated under varied values the modulated instrument parameter for each of the known compounds.

At operation 770, the spectral library is used by the analyte identification module 395 to predict compound identity of a test sample containing an unknown compound. At operation 780, the spectral library is further analyzed to build and/or improve a machine learning algorithm and train or improve the machine learning algorithm with the MS data from a plurality of known compounds stored in the spectral library. The trained machine learning algorithm may be used to determine the relationship between MS results and the instrument parameter at operation 750, to optimize instrument parameter or operating condition for analysis of an unknown compound, and/or to predict identify of the unknown compound at operation 770.

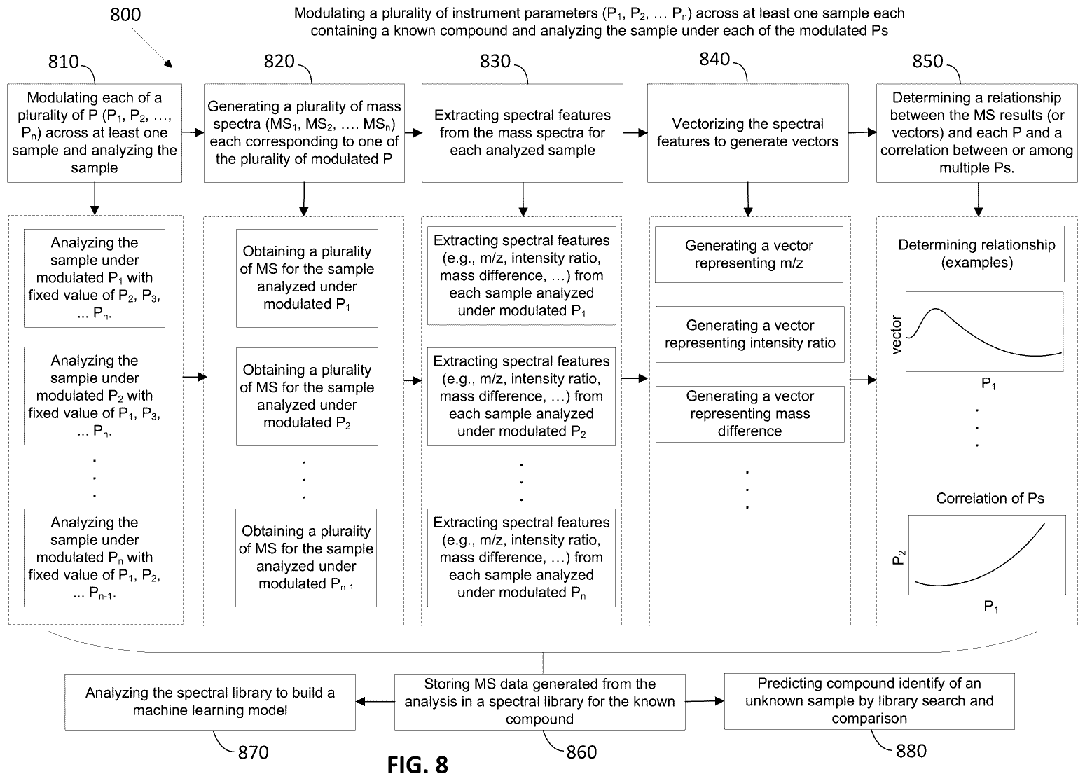

FIG. 8 illustrates a schematic view of a further exemplary workflow. The workflow 800 is a variation of the workflows 600 and 700 and is related to modulation of a plurality of instrument parameters (P₁, P₂, . . . . Pₙ) across at least one sample containing a known compound. In the illustrated example, the method 800 includes operations 810, 820, 830, 840, 850, 860, and optionally 870 and 880.

At operation 810, a plurality of instrument parameter (P=(P₁, P₂, P₃, . . . . Pₙ)) are each modulated and a plurality of values (V₁, V₂, V₃, . . . , Vₖ) varied and ramped in a range is set for each P across at least one sample. The at least one sample may include a single sample or multiple samples each containing a known compound. At operation 810, modulation is performed across the plurality of instrument parameters. For example, the sample is first analyzed under varied values of the modulated P₁ with a fixed value of other instrument parameters (P₂, P₃, . . . . Pₙ), then analyzed under varied values of the modulated P₂ with a fixed value of other instrument parameters (P₁, P₃, . . . . Pₙ), and continuously analyzed until the modulation of Pₙ is completed. At operation 820, a plurality of MS results (MS₁, MS₂, MS₃, . . . , MSₙ) each corresponding to the plurality of modulated P is generated. Each MSᵢ result may include a plurality mass spectra corresponding to the varied values of each modulated Pᵢ (i=1, 2, . . . , n). At operation 830, spectral features are extracted from the mass spectra with respect to each modulate Pᵢ (i=1, 2, . . . , n). At operation 840, the extracted spectral features are vectorized to generate a plurality of spectral vectors each representing the a specific spectral feature. At operation 850, the MS results are further processed to determine, for the known compound of the sample, a relationship between the spectral features and the varied values of each modulated Pᵢ, an impact of the modulation on each vector, an impact factor (significance) of the Pᵢ on each vector, or a correlation between or among different instrument parameters Pᵢ. At operation 860, the generated MS data and results are stored in a spectral library as a form of reference MS data for the known compound. The spectral library accordingly includes a plurality of reference mass spectra generated under each of the modulated instrument parameters for the known compound.

At operation 870, the spectral library is used by the analyte identification module 395 to predict compound identity of a test sample containing an unknown compound. At 880, the spectral library is further analyzed to build a machine learning algorithm and train or improve the machine learning algorithm with the stored MS data generated under a plurality of modulated instrument parameters. The trained machine learning algorithm may be used to determine the relationship between MS results and the instrument parameter at operation 750, to optimize multiple instrument parameters or operating conditions for analysis of an unknown compound, and/or to predict identify of the unknown compound at operation 870.

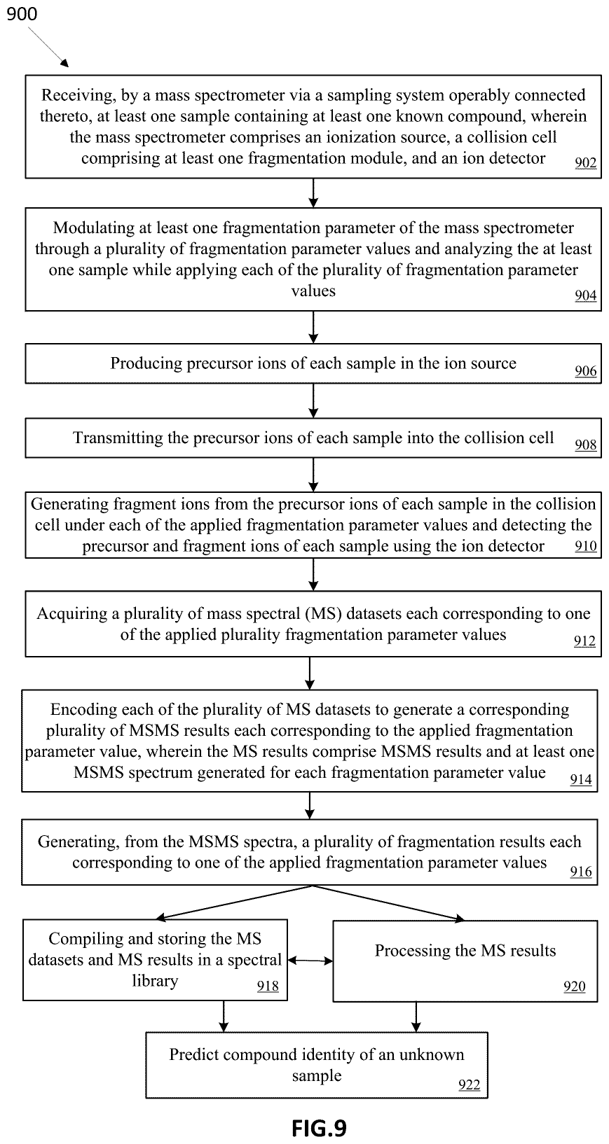

FIG. 9 illustrates a flow diagram of another example method for mass spectrometry, in particular, tandem mass spectrometry (MSMS). The method 900 may be performed through use of the system 10′ or the like in accordance with various embodiments described herein. In the illustrated example, method 900 includes at least one of operations 902, 904, 906, 908, 910, 912, 914, 916, 918, 920, and 922. At 902, a plurality of samples each containing at least one known compound is introduced through use of the sampling system 101 to the mass spectrometer 106. As described above, the mass spectrometer 106 includes an ion source 330, a collision cell 360 comprising at least one fragmentation module 370, a mass analyzer 340, and an ion detector 350. The plurality of sample is received by the ion source 330.

At operation 904, at least one fragmentation parameter related to the fragmentation module 370 is modulated. A particular fragmentation parameter is the collision energy (CE) applied to the precursor ions in the collision cell 360. The modulation may include setting a plurality of values varied or ramped in a range for the fragmentation parameter across at least one sample of the plurality of samples. The at least one sample may comprise a single sample or multiple samples of the plurality of samples. In some embodiments, operation 904 includes modulating at least one fragmentation parameter through a plurality of fragmentation parameter values and analyzing a single sample while applying each of the plurality of fragmentation parameter values. In some embodiments, operation 904 includes modulating at least one fragmentation parameter through a plurality of fragmentation parameter values and analyzing a plurality of samples while applying each of the plurality of fragmentation parameter values. In some embodiments, operation 904 includes modulating through a plurality of instrument parameter values while analyzing each sample of a plurality of samples.

Alternatively, if ramping is not an available option, the modulation may include varying the fragmentation module for the sample to be analyzed under a “single shot” of each fragmentation module. Non-limiting examples of the fragmentation module include collision induced dissociation (CID), surface induced dissociation (SID), electron capture dissociation (ECD), electron transfer dissociation (ETD), metastable-atom bombardment, photo-fragmentation.

At operation 906, precursor ions of each sample is produced in the ion source 330. At operation 908, the precursor ions of each sample is transmitted into the collision cell 360. At operation 910, the precursor ions of the sample in the collision cell are fragmented to generate fragment ions (product ions) under each of the applied fragmentation parameter values.

At operation 912, a plurality of MS datasets each corresponding to one of the applied plurality fragmentation parameter values. At operation 914, the plurality of MS datasets are encoded to generate a plurality of MS results each corresponding to the applied fragmentation parameter value, wherein the MS results comprise MSMS results and at least one MSMS spectrum generated for each of the plurality of fragmentation parameter values. At operation 916, a plurality of fragmentation results are generated from the MSMS spectra, with each fragmentation result corresponding to one of the applied fragmentation parameter values. Non-limiting examples of the fragmentation result include: total intensity of fragment ions, intensity ratio of fragment ions to precursor ions, extracted MSMS spectral features of the fragment ions, a MSMS spectral feature indicative of the precursor and fragment ions for each sample; a fragmentation pattern of each sample analyzed under the modulated fragmentation parameter; or a fragmentation pathway for the at least one known compound. Operation 916 may further include vectorizing the extracted MSMS Feature to generate at least one spectral vector that represent at least one MSMS feature. An example spectral vector is the total intensity of the fragment ions produced by the collision cell 360. A plurality of vectors may be generated at operation 916 to represent various MSMS features with respect to each analyzed sample.

At operation 918, the MS datasets and MSMS results generated from sample analysis are compiled and stored in a spectral library in a form of reference MSMS data. Accordingly, the spectral library may include a plurality of spectral entries, each spectral entry including the MS datasets, MSMS results, MSMS spectra, extracted MSMS features, vectors, etc., for each reference compound. The spectral library may advantageously provide a comprehensive profile of fragmentation patterns of a reference compound and the impact of fragmentation parameters on the fragmentation pattern.

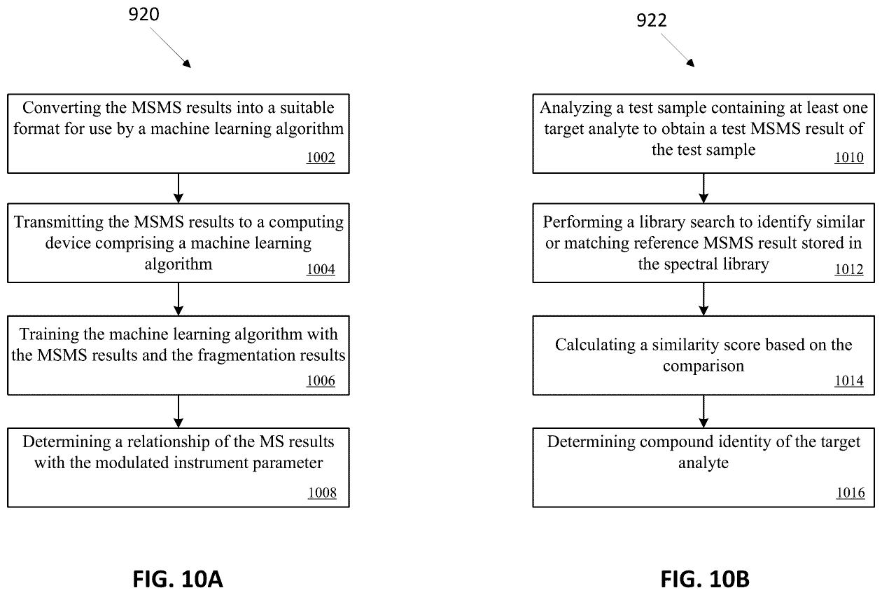

In a similar manner to the method 400, the method 900 may include operation 920 to process the MSMS results. At operation 920, the MS datasets and MSMS results corresponding to the modulated fragmentation parameters may be further processed through use of the data processing system 390. FIG. 10A illustrates an exemplary flow diagram of operation 920. In the illustrated example, operation 920 includes at least one of operations 1002, 1004, 1006, and 1008. At operation 1002, the MSMS results are converted into a suitable format for use by a machine learning algorithm. Operation 1004 includes transmitting the MSMS results in the suitable format to a computing device comprising a machine learning algorithm, e.g., the computing system 103 comprising the machine learning module 120. Operation 1006 includes training the machine learning algorithm with the MSMS results or building a machine learning algorithm. Operation 1008 includes determining a relationship of the MSMS results with the modulated instrument parameter, through use of the trained machine learning algorithm.

In a similar manner to the method 400, the method 900 may further include operation 922 to predict compound identity of an unknown sample using the spectral library containing reference MSMS results, through use of the analyte identification module 395. FIG. 10B illustrates a flow diagram of a particular example of operation 922. In the illustrated example, operation 922 includes at least one of operations 1010, 1012, 1014, and 1016. At operation 1010, a test sample containing at least one target analyte or an unknown compound of interest is analyzed by the mass spectrometer 106 containing the collision cell 360 to obtain a test MSMS result of the test sample. The test sample may be analyzed under at least one modulated fragmentation parameter (e.g., CE) to produce a plurality of test MSMS spectra obtained from each value of the modulated fragmentation parameter. At operation 1012, a library search is performed to compare the test MSMS spectra of the test sample to the reference MSMS spectra and fragmentation patterns of the reference compounds stored in the spectral library, with respect to each value of the modulated fragmentation parameter. At operation 1014, an overall similarity or matching score may be calculated based on the comparison at each value of the modulated fragmentation parameter. At operation 1016, a matching reference compound from the spectral library is identified based on the similarity score, and the identity of the unknown compound may be determined. Comparing with the traditional library search that disregards the impact of fragmentation parameter, the present spectral library takes the granularity of fragmentation parameters into account and provides a high data density for the reference MSMS data and fragmentation patterns. The present method thus enables a comprehensive comparison of the MSMS spectra and fragmentation patterns, increases dimensionality of spectral comparison in compound identification, and improves the confidence and accuracy of compound identification

Although various embodiments and examples are described herein, those of ordinary skill in the art will understand that many modifications may be made thereto within the scope of the present disclosure. Accordingly, it is not intended that the scope of the disclosure in any way be limited by the examples provided.

1. A method for mass spectrometry, the method comprising:
receiving, by a mass spectrometer via a sampling system operably connected thereto, at least one sample containing at least one known compound;
modulating at least one instrument parameter of the mass spectrometer through a plurality of instrument parameter values;
analyzing the at least one sample while applying each of the plurality of instrument parameter values;
acquiring a plurality of mass spectral (MS) datasets each corresponding to one of the applied plurality of instrument parameter values;
encoding each of the plurality of MS datasets to generate a corresponding plurality of MS results each corresponding to one of the applied instrument parameter values; and
compiling and storing the MS datasets and MS results in a spectral library in association with the applied instrument parameter values.

2-3. (canceled)

4. The method of claim 1, wherein the modulating the at least one instrument parameter comprises at least one of:
modulating through a plurality of instrument parameter values while analyzing a single sample; or
modulating through a plurality of instrument parameter values while analyzing a plurality of samples by setting a plurality of instrument parameter values ramped in a range for a single instrument parameter; or
modulating through a plurality of instrument parameter values while analyzing each sample of a plurality of samples, the at least one sample analyzed under at least one of the plurality of values.

5-7. (canceled)

8. The method of claim 7, further comprising:
transmitting at least a portion of the MS results to a computing device, the computing device comprising the machine learning algorithm and a processor for processing the MS results; and
training the machine learning algorithm with the MS results.

9. The method of claim 8 further comprising:
determining, for the at least one known compound, a relationship of the MS results with the at least one instrument parameter using the machine learning algorithm.

10. The method of claim 1, wherein:
the encoding further comprises:
extracting at least one spectral feature from the plurality of MS datasets; and
vectorizing the extracted spectral feature to generate at least one spectral vector; and
the method further comprises:
identifying a relationship between the at least one spectral vector and the at least one instrument parameter; or
determining an impact of the modulation on the spectral vector.

11. The method of claim 9, further comprising performing a principle component analysis (PCA) on the at least one spectral vector.

12. The method of claim 9, wherein the MS results comprise a mass spectra generated for each instrument parameter value, and wherein the extracting at least one spectral feature comprises at least one of:
calculating the total ion intensity of the mass spectra;
annotating/identifying/grouping MS peaks of the mass spectra;
calculating m/z values, peak area, and intensities of MS peaks;
determining a relationship between related MS peaks;
extracting a spectral feature indicative of a fragmentation pattern;
identifying precursor ions and product ions; or
extracting a spectral feature indicative of a sample matrix.

13. The method of claim 1, further comprising:
analyzing a test sample containing at least one target analyte to obtain a test result of the test sample;
comparing the test result with the spectral library; and
predicting an identity of the at least one analyte in the test sample.

14-15. (canceled)

16. The method of claim 1, wherein:
the sampling system comprises an Acoustic Droplet Ejector (ADE) operably coupled to an Open Port Interface (OPI); and
each of the plurality of samples is ejected from a sample volume by the ADE and introduced to the mass spectrometer through the OPI.

17. The method of claim 1, wherein the at least one instrument parameter comprises at least one of:
a collision energy (CE);
an electron energy;
a parameter related to fragmentation;
a parameter related to ionization;
a parameter related to the introduction of ions to a quadrupole ion guide; or
a parameter that controls an ion mobility device.

18. The method of claim 1, wherein:
the mass spectrometer comprises an ionization source, a collision cell, and an ion detector;
the collision cell comprises at least one fragmentation module selected from: collision induced dissociation (CID), surface induced dissociation (SID), electron capture dissociation (ECD), electron transfer dissociation (ETD), metastable-atom bombardment, photo-fragmentation, or combinations thereof;
the at least one instrument parameter comprises a fragmentation parameter that controls the fragmentation module, and
the plurality of instrument parameter values comprises a plurality of fragmentation parameter values;
the method further comprising:
producing precursor ions of each sample in the ionization source;
transmitting the precursor ions of each sample into the collision cell;
generating fragment ions from the precursor ions of each sample in the collision cell under each of the applied modulated fragmentation parameter; and
detecting the precursor and fragment ions using the ion detector,
wherein the MS results comprise at least one MSMS spectrum generated for each fragmentation parameter value.

19-20. (canceled)

21. The method of claim 20, further comprising generating, from the MSMS spectra, a plurality of fragmentation results each corresponding to one of the applied fragmentation parameter values, wherein the plurality of fragmentation results comprise at least one of:
a spectral feature indicative of the precursor and fragment ions for each sample;
a fragmentation pattern of each sample analyzed under each one of the applied fragmentation parameter values; or
a fragmentation pathway for the at least one known compound.

22. The method of claim 20, further comprising:
training a machine learning algorithm with the plurality of fragmentation results; and
determining, for the at least one known compound, a relationship of the plurality of fragmentation results with the fragmentation parameter, using the machine learning algorithm.

23. A method for mass spectrometry, the method comprising:
generating a plurality of MS results for a plurality of known compounds by repeating the method according to claim 1 or an operation thereof; and
compiling and storing the plurality of MS results corresponding to the plurality of known compounds in the spectral library.

24. A method for mass spectrometry of claim 1, further comprising:
converting the MS results in a suitable format for use by a machine learning algorithm;
transmitting at least a portion of the MS results to a computing device, the computing device comprising the machine learning algorithm and a processor for processing the MS results; and
determining, for the at least one known compound, a relationship of the MS results with the modulated instrument parameter, using the machine learning algorithm,
wherein the corresponding plurality of MS results each correspond to one of the applied instrument parameter values.

25. (canceled)

26. A method for mass spectrometry, comprising:
receiving, by a mass spectrometer via a sampling system operably connected thereto, at least one sample containing at least one known compound, wherein the mass spectrometer comprises an ionization source, a collision cell comprising at least one fragmentation module, and an ion detector;
modulating at least one fragmentation parameter of the mass spectrometer through a plurality of fragmentation parameter values;
analyzing the at least one sample while applying each of the plurality of fragmentation parameter values;
producing precursor ions of each sample in the ionization source;
transmitting the precursor ions of each sample into the collision cell;
generating fragment ions from the precursor ions of each sample in the collision cell under each of the applied fragmentation parameter values;
detecting the precursor and fragment ions of each sample using the ion detector;
acquiring a plurality of mass spectral (MS) datasets each corresponding to one of the applied plurality fragmentation parameter values;
encoding each of the plurality of MS datasets to generate a corresponding plurality of MS results each corresponding to the applied fragmentation parameter value, wherein the MS results comprise at least one MSMS spectrum generated for each instrument parameter value;
generating, from the MSMS spectra, a plurality of fragmentation results each corresponding to one of the applied fragmentation parameter values; and
compiling and storing the MS datasets and MS results in a spectral library in association with the applied fragmentation parameter values.

27. A system comprising:
a mass spectrometer;
a high-throughput sampling system operative to introduce a plurality of samples to the mass spectrometer;
a processor operatively coupled to the high-throughput sampling system and the mass spectrometer; and
memory, coupled to the processor, the memory storing instructions that, when executed by the processor, perform operations comprising the method of claim 1.

28. A computer program product, comprising a non-transitory computer-readable storage medium whose contents include a program with instructions being executed on a processor so as to perform the method of claim 26, the method further comprising determining, for the at least one known compound, a relationship of the MS results with the modulated instrument parameter.

29-30. (canceled)

31. The computer program product of claim 28, wherein the method further comprises training the machine learning algorithm with the MS results.

32. A method for mass spectrometry, the method comprising:
receiving, by a mass spectrometer via a sampling system operably connected thereto, at least one sample containing at least one known compound;
modulating at least one instrument parameter of the mass spectrometer through a plurality of instrument parameter values;
analyzing the at least one sample while applying each of the plurality of instrument parameter values;
acquiring a plurality of mass spectral (MS) datasets each corresponding to one of the applied plurality of instrument parameter values for the at least one known compound;
encoding each of the plurality of MS datasets to generate a corresponding plurality of MS results each corresponding to one of the applied instrument parameter values;
analyzing a test sample containing at least one target analyte to obtain a test result of the test sample;
comparing the test result with the MS results of the at least one known compound; and
predicting an identity of the at least one analyte in the test sample.
